(19) United States
(12) Patent Application Publication
(10) Pub. No.: US 2021/0030544 A1
(43) Pub. Date: Feb. 4, 2021

(54) PENILE IMPLANT OUTER SUPPORT STRUCTURE WITHIN NEOPHALLUS FOR NEOPHALLUS SURGERY

(71) Applicant: Boston Scientific Scimed, Inc., Maple Grove, MN (US)

(72) Inventors: Jessica Elizabeth Felton, Minneapolis, MN (US); Randall Paul Rowland, Eden Prairie, MN (US); Paul John Gindele, Buffalo, MN (US); Aaron Roydon Johnson, Minneapolis, MN (US); Abigail Rae Brooks, Hopkins, MN (US); Nickolas Dalbec, Edina, MN (US); Bernard Andre Wasscher, Mound, MN (US)

(21) Appl. No.: 16/947,353

(22) Filed: Jul. 29, 2020

Related U.S. Application Data
(60) Provisional application No. 62/881,060, filed on Jul. 31, 2019.

(51) Int. Cl. A61F 2/26

(52) U.S. Cl. CPC A61F 2/26; A61F 2250/0051

(57) ABSTRACT

An implantable device for penile construction includes a tissue ingrowth cover. The tissue ingrowth cover may be disposed between a penile prosthesis and an interior wall of a neophallus, when the penile prosthesis is implanted within the neophallus.

### CROSS-REFERENCE TO RELATED APPLICATION

This application claims priority to U.S. Provisional Patent Application No. 62/881,060, filed on Jul. 31, 2019, entitled “PENILE IMPLANT OUTER SUPPORT STRUCTURE WITHIN NEOPHALLUS FOR NEOPHALLUS SURGERY”, which is incorporated by reference herein in its entirety.

### TECHNICAL FIELD

This disclosure relates generally to penile construction.

### BACKGROUND

In some examples, penile prostheses for erectile dysfunction have been used off-label in penile construction or reconstruction cases such as female to male (FTM) transgender individuals, and natal males with penile reconstruction (e.g., congenital disorders, amputation (penile cancer), trauma, etc.). For example, a patient undergoes a phalloplasty procedure (e.g., single stage or multiple stages) in which a neophallus is surgically constructed from tissue grafts taken from other parts of the body.

The creation of a penis generally involves the creation of a tube within a tube. The inside tube is the urethra, the structure through which men urinate. The outside tube is the penile shaft. This structure, known as the neophallus (neo- for new), is surgically attached to the pelvis. The urethra of the neophallus is connected to the existing urethra. The blood vessels and nerves are attached to those of the pelvis. The exterior of the neophallus is then sculpted to resemble a penis shaft and head.

Since the neophallus is made of skin and does not contain the erectile tissues of a biological penis, the neophallus does not have the ability to achieve erection. As referenced above, penile prostheses have been developed to enable patients to have intercourse with a partner, and versions of these prostheses have been used in both natal males and FTM individuals.

Current production and surgical techniques for making and implanting such penile prostheses provide best-available options for candidate patients, but do not provide results fully matching natural penile appearance and function. Moreover, the current surgical techniques are often complex and/or lengthy, and are prone to infection, scarring, and other difficulties. As a result, significant areas of improvement exist for increasing patient satisfaction in the area of neophallus surgery.

### SUMMARY

According to an aspect, an implantable device for penile construction includes at least one tissue ingrowth cover configured to be disposed between a penile prosthesis and an interior wall of a neophallus, when the penile prosthesis is implanted within the neophallus.

According to an aspect, an implantable device for penile construction includes a penile prosthesis device including at least one inflatable member, and a pump assembly for inflating the at least one inflatable member. The implantable device further includes at least one tissue ingrowth cover configured to be disposed between the at least one inflatable member and an interior wall of a neophallus, when the at least one inflatable member is implanted within the neophallus.

According to an aspect a method of implanting an implantable device for penile construction includes disposing at least one tissue ingrowth cover over at least a portion of a penile prosthesis. The method further includes implanting the at least one tissue ingrowth cover and the penile prosthesis within a neophallus, with the at least one tissue ingrowth cover positioned between the penile prosthesis and an interior wall of the neophallus.

### BRIEF DESCRIPTION OF THE DRAWINGS

FIG. 1 illustrates a tissue ingrowth cover for a penile prosthesis, according to an aspect.

FIG. 2 illustrates the tissue ingrowth cover of FIG. 1 as a separate tissue ingrowth sheath for a penile prosthesis, according to an aspect.

FIG. 3 illustrates the tissue ingrowth cover of FIG. 1 as including a proximal cover and a distal cover disposed at a proximal end and distal end, respectively, of a penile prosthesis, according to an aspect.

FIG. 4 illustrates the tissue ingrowth cover of FIG. 1 covering essentially all of a penile prosthesis, according to an aspect.

FIG. 5 illustrates a cross section of a neophallus with a tissue ingrowth cover included, according to an aspect.

FIG. 6 illustrates a penile prosthesis device, according to an aspect.

### DETAILED DESCRIPTION

Detailed embodiments are disclosed herein. However, it is understood that the disclosed embodiments are merely examples, which may be embodied in various forms. Therefore, specific structural and functional details disclosed herein are not to be interpreted as limiting, but merely as a basis for the claims and as a representative basis for teaching one skilled in the art to variously employ the embodiments in virtually any appropriately detailed structure. Further, the terms and phrases used herein are not intended to be limiting, but to provide an understandable description of the present disclosure.

The terms “a” or “an,” as used herein, are defined as one or more than one. The term “another,” as used herein, is defined as at least a second or more. The terms “including” and/or “having”, as used herein, are defined as comprising (i.e., open transition). The term “coupled” or “moveably coupled,” as used herein, is defined as connected, although not necessarily directly and mechanically.

In general, the embodiments are directed to bodily implants, and methods of making and/or surgically implanting such bodily implants. The term patient or user may hereafter be used for a person who benefits from the medical device or the methods disclosed in the present disclosure. For example, the patient can be a person whose body is implanted with the medical device or the method disclosed for operating the medical device by the present disclosure.

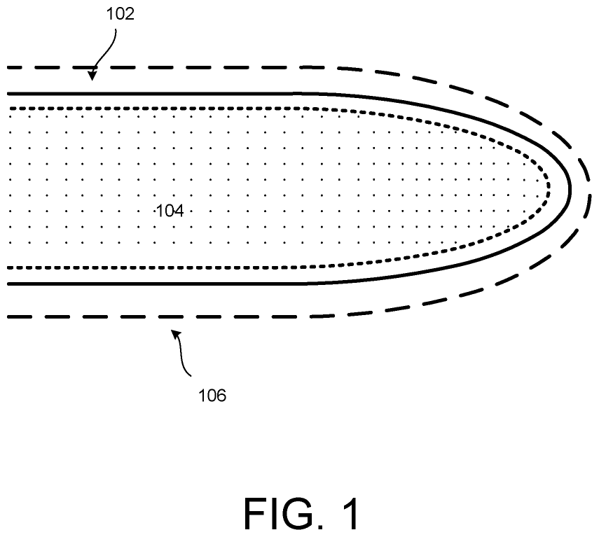

FIG. 1 illustrates a tissue ingrowth cover 102 for a penile prosthesis 104, according to an aspect. As described in detail, below, the tissue ingrowth cover 102 provides a structure that is located between at least a portion of the penile prosthesis 104 and a neophallus 106. That is, the tissue ingrowth cover 102 is positioned between one or more portions of the penile prosthesis 104 and an interior wall of the neophallus 106.

As further explained below, the tissue ingrowth cover 102 provides a structure that promotes tissue ingrowth, simulates characteristics and aspects of natural penile function, fills a void between the penile prosthesis 104 and an interior wall of the neophallus 106, acts as a bulking agent, anchor the penile prosthesis 104 to a body of the patient, and otherwise contributes to successful and satisfactory patient outcomes. The tissue ingrowth cover 102 may be exterior to (e.g., separate from) the penile prosthesis 104, or may be formed as part of the penile prosthesis 104. The tissue ingrowth cover 102 may be implanted at a time of creation of the neophallus 106, or at a time of implant of the penile prosthesis 104 into the neophallus 106 (which may be particularly suitable for embodiments in which the tissue ingrowth cover 102 is formed as part of the penile prosthesis 104).

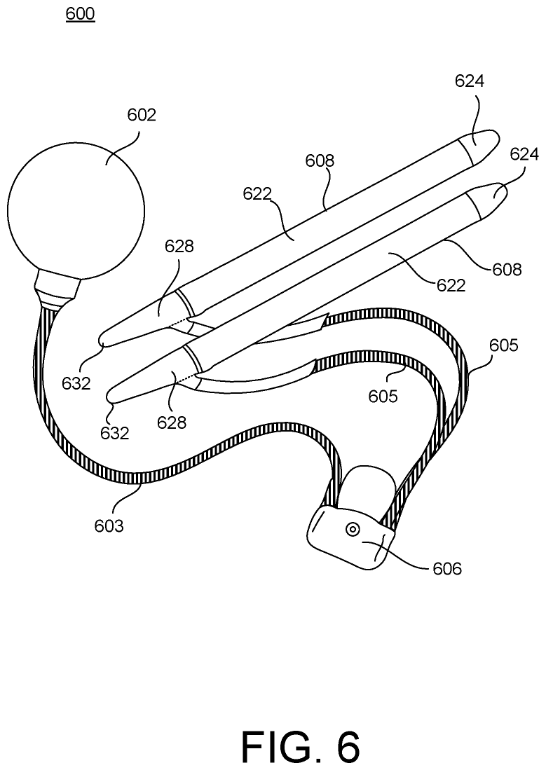

As described and illustrated below, e.g., with respect to FIG. 6, the penile prosthesis 104 may be part of a penile prosthetic device, or penile prosthetic system, that may include various other components. For purposes of the present description, the term penile prosthesis refers to an elongated member or shaft, e.g., formed as a cylinder and inserted into the neophallus 106 as part of a penile prosthetic device or system. Thus, for example, and as described below in various examples, such a penile prosthetic device or system may include more than one penile prosthesis, e.g., may include two penile prostheses inserted into the single neophallus 106.

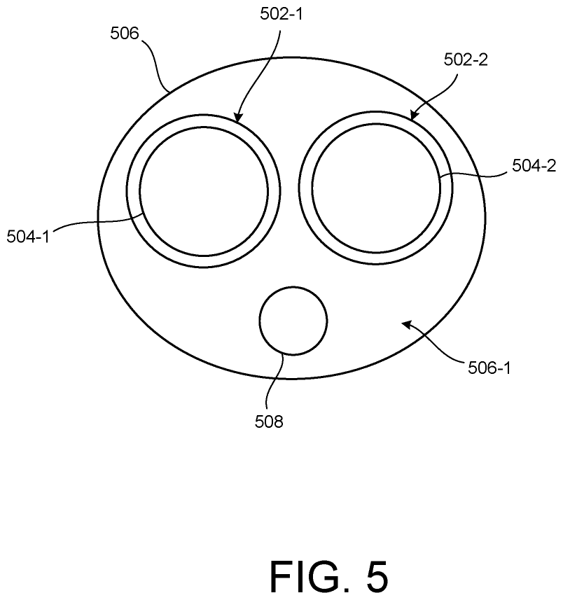

In some examples, the penile prosthesis 104 is configured to be inserted into the neophallus 106, e.g., a newly constructed shaft of skin in the shape of a penis, where the neophallus 106 is formed from tissue donated from a body part (e.g., thigh, forearm) of the patient. Thus, in some examples, the penile prosthesis 104 is a neophallus implant. In some examples, the penile prosthesis 104 is a penile prosthesis originally designed for erectile dysfunction. In some examples, the penile prosthesis 104 is an adjustable member configured to increase in length over time to assist with the creation of a neophallus. In some examples, the penile prosthesis 104 is a penile shaft that defines one or more lumens (or a solid core), where the penile shaft is configured to accommodate a neourethra within the neophallus 106 (an example of which is illustrated in FIG. 5). In some examples, the penile prosthesis 104 is a hydraulic penile prosthesis in which the penile prosthesis 104 is an inflation member (e.g., a single cylinder) that receives fluid from a fluid reservoir via a pump assembly. In some examples, the penile prosthesis 104 is a malleable penile prosthesis in which the penile prosthesis is malleable. In some examples, the penile prosthesis 104 is a non-hydraulic mechanical penile prosthetic device.

As referenced and described, the devices and techniques described herein may be used for female to male (FTM) surgery, or for natal males undergoing penile construction (or reconstruction). However, there may be difficulties associated with how the penile prosthesis 104 is attached to the pelvic region of the patient. In natal males, the proximal ends of the corpora cavernosa tunnel deep into the pelvic region, and, in some examples, provide the cavity in which two penile prostheses (e.g., two cylinders) are disposed, as well as a way to prevent migration or crossover of the cylinders. Examples of such a two-cylinder approach are illustrated and described below, e.g., with respect to FIGS. 5 and 6.

In the natal males undergoing penile reconstruction due to amputation, the proximal corpora may still be intact, and may serve to anchor the proximal ends of the penile prosthesis dual cylinders. However, in female-to-male (FTM) transgender individuals, natal males with birth defects, and/or severe trauma cases, these features of the proximal corpora may not be present in the same manner, and, there may be difficulties with anchoring these devices to the pelvic region (e.g., pelvic bone), and/or with utilizing the two-cylinder approach in general. As described and illustrated below, the tissue ingrowth cover 102 provides or facilitates anchoring of the penile prosthesis 104, by enabling tissue ingrowth in a manner that is easy, efficient, and consistent for surgeons to implement. The tissue ingrowth cover 102 also facilitates implementation of the two-cylinder approach, by providing a buffer(s) between the two cylinders, and generally providing structure stability and reducing or eliminating cylinder migration or crossover.

In some examples, the tissue ingrowth cover 102 includes tissue ingrowth materials, such as hydroxyapatite, or porous polymer scaffolds. In some examples, the tissue ingrowth cover 102 includes, or uses, cadaveric bone, animal biologic tissues, and materials known for tissue ingrowth properties. As described, such material(s) may be used to help fuse and anchor the tissue ingrowth cover 102, and thus the penile prosthesis 104, to the patient. In additional or alternative implementations, porous, hydroxyapatite-type material for bone/tissue ingrowth, may be used, and/or pre-formed open cell foam matrix for tissue ingrowth. Also, a portion of the tissue ingrowth cover may be biodegradable to invoke the production of scar tissue while allowing the device to be removable for revision surgery.

In further additional or alternative implementations, the tissue ingrowth cover 102 may include suitable textile(s), such as woven fabric or knit mesh. The tissue ingrowth cover 102 may include, or use, porous plastic materials, such as ePTFE. The types of animal biologic materials referenced above may include, in some examples, one or more of collagen, bovine pericardium, or porcine dermis.

The tissue ingrowth cover 102 may be blended with non-tissue ingrowth material, e.g., silicone, to specify areas that will have tissue ingrowth (as compared to areas that should not have tissue ingrowth). The tissue ingrowth cover 102 may also be embedded or impregnated with substances that are anti-microbial, biodegradable, improve tissue ingrowth, and/or promote healing.

In providing anchoring as referenced above, the tissue ingrowth cover 102 may minimize or eliminate a need for use of a mechanical anchor element(s) (e.g., bone screws). In addition to providing such anchoring and other functionalities described above, the tissue ingrowth cover 102 may serve to prevent erosion, while providing improved structural integrity to the neophallus 106 as a whole (e.g., as compared to a single cylinder penile implant). The tissue ingrowth cover 102 may further serve to reduce and distribute load transfer from the neophallus 106 during use, while improving an aesthetic appearance (e.g., additional girth) of the neophallus 106, as well as providing improved palpability (e.g., more natural feel).

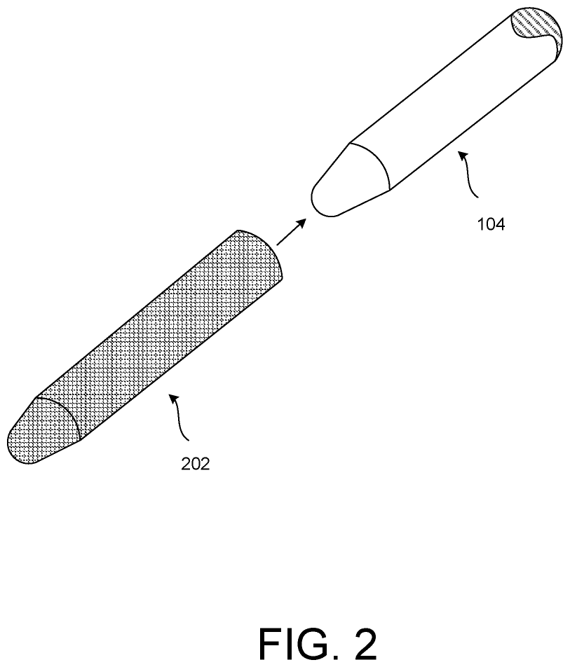

FIG. 2 illustrates the tissue ingrowth cover 102 of FIG. 1 as a separate tissue ingrowth sheath 202 for the penile prosthesis 104, according to an aspect. As illustrated, the tissue ingrowth sheath 202 is a separate structure from the penile prosthesis 104 that fits over, and receives, the penile prosthesis 104.

In the example of FIG. 2, the tissue ingrowth sheath 202 may be manufactured to accommodate a specific size, shape or other characteristic(s) of the penile prosthesis 104 to be used in a particular surgery. Thus, a surgeon may easily and reliably insert the penile prosthesis 104 into the tissue ingrowth sheath 202 during surgery, so that a length of the surgery is not adversely impacted, or is improved, over existing surgical techniques.

Further, as a separate structure, the tissue ingrowth sheath 202 also may be removed from the penile prosthesis in an easy and efficient manner. For example, a surgeon may remove the tissue ingrowth sheath 202 to use a different implementation of the tissue ingrowth sheath 202, if desired. Further, if necessary, it is straightforward to execute a replacement of the tissue ingrowth sheath 202 within the neophallus 106, e.g., during a revision surgery.

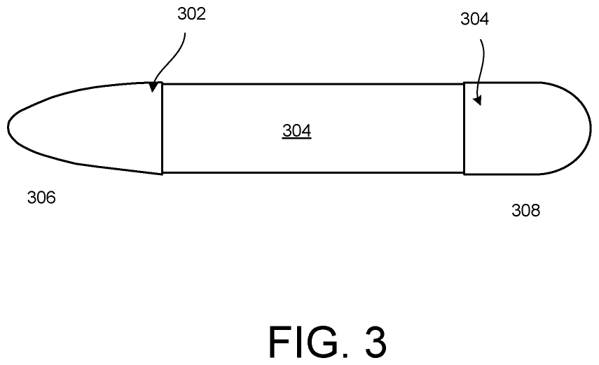

In various implementations, the tissue ingrowth sheath 202 of FIG. 2 may extend over essentially an entirety of the penile prosthesis 104, or over any specified portion thereof. Further, as may be appreciated from the below description and illustration of FIG. 3, the tissue ingrowth sheath 202 may be implemented using more than one tissue ingrowth sheath. For example, a first (proximal) tissue ingrowth sheath may be placed over a proximal end of the penile prosthesis 104, and a second (distal) tissue ingrowth sheath may be placed over a distal end of the penile prosthesis 104.

Specifically, FIG. 3 illustrates the tissue ingrowth cover 102 of FIG. 1, which may represent the tissue ingrowth sheath 202 of FIG. 2, as including a proximal cover 302 and a distal cover 304, disposed at a proximal end 306 and a distal end 308, respectively, of the penile prosthesis 104, according to an aspect. As just referenced, the proximal cover 302 and the distal cover 304 may represent sheaths, consistent with the example of FIG. 2.

In some examples, the proximal end 306 is a rear tip of a penile implant, while the distal end is a front tip of the penile implant. In some examples, the proximal end 306 may define part of an attachment interface to couple the penile prosthesis 104 to a pelvic bone of a patient.

In some example implementations, one or both of the proximal cover 302 and the distal cover 304 may be implemented as a part of, e.g., integrally, with the penile prosthesis 104. For example, one or both of the proximal cover 302 and the distal cover 304 may be coupled to the penile prosthesis 104 at a time of manufacture thereof. Various embodiments may also have any combination of the described example implementations. For example, a tissue ingrowth cover(s) may be integral at some localized portions of the penile prosthesis 104, while other tissue ingrowth cover(s) may be implemented as removable sheath(s). In general, example implementations of one or more tissue ingrowth covers may cover all, or any desired portions or percentages of the penile prosthesis 104.

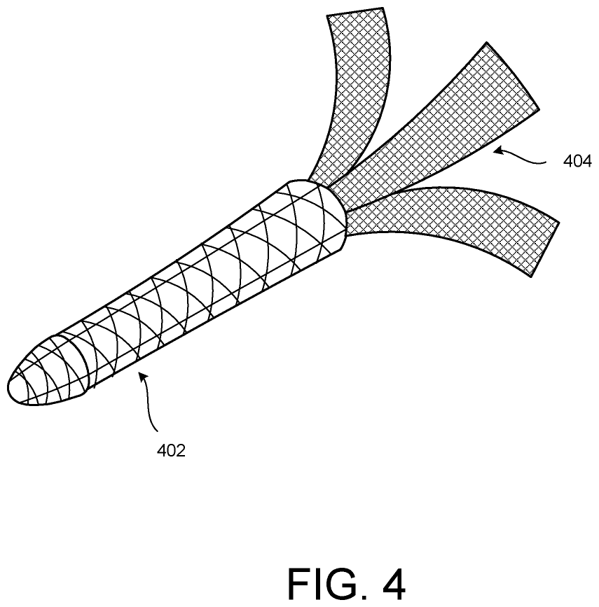

FIG. 4 illustrates a tissue ingrowth cover 402 covering essentially all of a penile prosthesis 404, according to an aspect. That is, as illustrated, the tissue ingrowth cover 402 runs along an entire length of the pictured prosthetic device.

FIG. 5 illustrates a cross section of a neophallus with a tissue ingrowth cover included, according to an aspect. The example of FIG. 5 illustrates a dual-cylinder implementation of a penile prosthesis, designed to more closely mimic natural penile anatomy.

Specifically, FIG. 5 illustrates a cross-section of a neophallus 506 showing a penile prosthesis 504-1 and a penile prosthesis 504-2, and with a neourethra 506. The penile prosthesis 504-1 is covered in the cross section with a corresponding tissue ingrowth cover 502-1, while the penile prosthesis 504-2 is covered in the cross section with a corresponding tissue ingrowth cover 502-2. A neourethra 508 is also included.

As referenced above, such a dual-cylinder approach more closely mimics natural male anatomy, which includes two corpora of the corpora cavernosa. Natural penile anatomy also includes corpus spongiosum, tunica albuginea, and other erectile tissues, structures, or aspects.

However, the neophallus 506 typically includes a rolled skin flap(s) with an interior 506-1 that, includes, e.g., fat and connective tissues. As a result, if a dual-cylinder approach is attempted in the context of conventional neophallus surgery, the two cylinders are prone to crossing and/or migration, when inflated or deflated, which may lead to malfunctions and other undesirable outcomes. Therefore, conventional approaches to neophallus surgery with penile prostheses often include only a single cylinder, including, if necessary, removal of one of two cylinders that may be included in a manufactured penile prosthetic system. Such single cylinder approaches may result in undesired or suboptimal characteristics of the resulting neophallus, including, e.g., with respect to size and rigidity of the resulting neophallus.

As may be understood from the present description, and from the illustration of FIG. 5, inclusion of tissue ingrowth covers 502-1 and 502-2 addresses the above and other difficulties and enables the illustrated dual-cylinder approach. For example, the tunica albuginea refers to a fibrous layer of connective tissue that surrounds and supports the corpora cavernosa of the penis. In FIG. 5, the tissue ingrowth covers 502-1 and 502-2 surround and support the dual cylinders 504-1 and 504-2 of the penile prosthesis device and prevent crossover and migration of the cylinders 504-1 and 504-2. Thus, the example embodiment of FIG. 5 is able to provide the advantages of a dual-cylinder approach, as referenced above.

FIG. 6 illustrates a penile prosthesis device, according to an aspect. In the example of FIG. 6, the penile prosthesis device 600 may be an inflatable penile prosthesis device. The inflatable penile prosthesis 600 may be an example of any of the implantable devices discussed herein and may include any of the features discussed with reference to the previous figures.

The penile prosthesis device 600 may include a pair of cylinders 608, and the pair of cylinders or inflatable members 608 are configured to be implanted in a pelvic region. For example, one or both of the cylinders 608 may be coupled to a suitable anchor plate. The cylinder 608 may include a first end portion 624, a cavity or inflation chamber 622, and a second end portion 628 having a rear tip 632.

It will be appreciated that any one or more of the various examples of tissue ingrowth cover(s) 102, 202, 302, 402, and 502-1/502-2 of preceding FIGS. 1-5 may be incorporated in, or used with, the example of FIG. 6. Thus, for example, the second end portion 628 may be observed to correspond to, or provide an example of, the proximal end 306 of FIG. 3. Similarly, the first end portion 624 may be observed to correspond to, or provide an example of, the distal end 308 of FIG. 3. Further, in the example of FIG. 5, the dual cylinders 608 may be understood to be configured to represent the cylinders 504-1, 504-2.

A pump assembly 606 may be implanted into the patient's scrotum. A pair of conduit connectors 605 may attach the pump assembly 606 to the pair of inflatable members or cylinders 608 such that the pump assembly 606 is in fluid communication with the pair of inflatable members or cylinders 608. Also, the pump assembly 606 may be in fluid communication with the fluid reservoir 602 via a conduit connector 603. The fluid reservoir 602 may be implanted into the patient's abdomen. The inflation chamber or portion 622 of the cylinder 608 may be disposed within the neophallus. The first end portion 624 of the cylinder 608 may be at least partially disposed within the glans portion of the neophallus. The second end portion 628 may be implanted into the patient's pubic region with the rear tip 632 configured to be attached to an anchor plate or other suitable connecting member(s).

The patient may operate the pump assembly 606 to start an inflation mode, where the pump assembly 606 is configured to facilitate the transfer of fluid from the fluid reservoir 602 to the cylinders 608. In some examples, when the user switches to the deflation mode, at least some of the fluid can automatically be transferred back to the fluid reservoir 602 (due to the difference in pressure from the cylinders 608 to the fluid reservoir 602).

While certain features of the described implementations have been illustrated as described herein, many modifications, substitutions, changes and equivalents will now occur to those skilled in the art. It is, therefore, to be understood that the appended claims are intended to cover all such modifications and changes as fall within the scope of the embodiments.

What is claimed is:

1. An implantable device for penile construction, the implantable device comprising:
at least one tissue ingrowth cover configured to be disposed between a penile prosthesis and an interior wall of a neophallus, when the penile prosthesis is implanted within the neophallus.

2. The implantable device of claim 1, wherein the at least one tissue ingrowth cover includes a tissue ingrowth sheath that is separate from the penile prosthesis and shaped to fit over the penile prosthesis.

3. The implantable device of claim 1, wherein the at least one tissue ingrowth cover is integral with the penile prosthesis.

4. The implantable device of claim 1, further comprising a first cylinder and a second cylinder of the penile prosthesis, wherein the at least one tissue ingrowth cover includes a first tissue ingrowth cover configured to be disposed over at least a portion of the first cylinder, and a second tissue ingrowth cover configured to be disposed over at least a portion of the second cylinder.

5. The implantable device of claim 4, wherein the first tissue ingrowth cover and the second tissue ingrowth cover are positioned to prevent crossover of the first cylinder and the second cylinder within neophallus.

6. The implantable device of claim 1, wherein the at least one tissue ingrowth cover is configured to be disposed at a proximal end of the penile prosthesis.

7. The implantable device of claim 1, wherein the at least one tissue ingrowth cover is configured to promote tissue ingrowth with respect to a pelvic region and thereby facilitate anchoring of the penile prosthesis to the pelvic region.

8. The implantable device of claim 1, wherein the at least one tissue ingrowth cover is configured to be disposed at a distal end of the penile prosthesis.

9. The implantable device of claim 1, wherein the at least one tissue ingrowth cover is formed using one or more of: hydroxyapatite, porous plastic, animal biologic tissues, a textile, and a pre-formed open cell foam matrix.

10. The implantable device of claim 1, wherein the at least one tissue ingrowth cover is formed using one or more animal biologic tissues including one or more of: cadaveric bone, collagen, bovine pericardium, or porcine dermis.

11. The implantable device of claim 1, wherein the at least one tissue ingrowth cover is blended with non-tissue ingrowth material.

12. The implantable device of claim 11, wherein the non-tissue ingrowth material includes silicone.

13. The implantable device of claim 1, wherein the at least one tissue ingrowth cover is blended with non-tissue ingrowth material that includes one or more substance that is anti-microbial.

14. The implantable device of claim 1, further comprising a single inflatable member of the penile prosthesis that is covered at least partially by the at least one tissue ingrowth cover.

15. A method of implanting an implantable device for penile construction, the method comprising:
disposing at least one tissue ingrowth cover over at least a portion of a penile prosthesis; and
implanting the at least one tissue ingrowth cover and the penile prosthesis within a neophallus, with the at least one tissue ingrowth cover positioned between the penile prosthesis and an interior wall of the neophallus.

16. The method of claim 15, further comprising:
disposing a first tissue ingrowth cover over at least a portion of a first cylinder;
disposing a second tissue ingrowth cover over at least a portion of a second cylinder; and
implanting the first cylinder and the first tissue ingrowth cover, and the second cylinder and the second tissue ingrowth cover, within the neophallus.

17. The method of claim 15, wherein the disposing the at least one tissue ingrowth cover further comprises:
disposing a tissue ingrowth sheath that is separate from the penile prosthesis and shaped to fit over the penile prosthesis.

18. An implantable device for penile construction, the implantable device comprising:
a penile prosthesis device including at least one inflatable member, and a pump assembly for inflating the at least one inflatable member; and
at least one tissue ingrowth cover configured to be disposed between the at least one inflatable member and an interior wall of a neophallus, when the at least one inflatable member is implanted within the neophallus.

19. The implantable device of claim 18, the penile prosthesis further comprising a second inflatable member, wherein the at least one tissue ingrowth cover includes a second tissue ingrowth cover configured to be disposed between the second inflatable member and the interior wall of a neophallus, when the second inflatable member is implanted within the neophallus.

20. The implantable device of claim 18, wherein the at least one tissue ingrowth cover includes a first tissue ingrowth cover disposed at a proximal end of the at least one inflatable member, and a second tissue ingrowth cover disposed at a distal end of the at least one inflatable member.
